(19) United States
(12) Patent Application Publication
(10) Pub. No.: US 2007/0185194 A1
(43) Pub. Date: Aug. 9, 2007

(54) Stable oral compositions of azithromycin monohydrate

(76) Inventors: Kamal Mehta, Rajasthan (IN); Rajeev Shankar Mathur, Gurgaon (IN); Sujata Paul, West Bengal (IN); Sanjeev Kumar Sethi, Uttar Pradesh (IN); Rajiv Malik, Delhi (IN)

Correspondence Address: RANBAXY INC., 600 COLLEGE ROAD EAST, SUITE 2100, PRINCETON, NJ, 08540, US

(21) Appl. No.: 10/561,827

(22) PCT Filed: Jul. 1, 2004
(86) PCT No.: PCT/IB04/02191

§ 371 Date: Nov. 27, 2006

(30) Foreign Application Priority Data
Jul. 1, 2003 (IN) 861/DEL/2003

(51) Int. Cl. A61K 31/35

(52) U.S. Cl. 514/460

(57) ABSTRACT

The present invention relates to stable oral compositions of azithromycin monohydrate with reduced bitterness, processes for making these compositions, and methods of using these compositions for the treatment of microbial infections. The stable oral compositions of azithromycin include an azithromycin premix, at least one pharmaceutically accepted excipient, and, optionally, at least one taste-masking agent. The azithromycin premix includes azithromycin monohydrate and at least one additive.

### FIELD OF THE INVENTION

The present invention relates to stable oral compositions of azithromycin monohydrate with reduced bitterness, processes for making these compositions, and methods of using these compositions for the treatment of microbial infections.

### BACKGROUND OF THE INVENTION

Azithromycin is the U.S.A.N. (generic name) for 9α-aza-9α-methyl-9-deoxo-9α-homoerythromycin A, a broad-spectrum antimicrobial compound derived from erythromycin A. Azithromycin is described in U.S. Pat. No. 4,474,768 and Kobrehel et al., U.S. Pat. No. 4,517,359. These patents disclose that azithromycin and certain derivatives thereof possess antibacterial properties and are accordingly useful as antibiotics. Azithromycin is indicated for infections caused by susceptible organisms in lower respiratory tract infections including bronchitis and pneumonia, skin and soft tissue infections, otitis media and in upper respiratory tract infections including sinusitis and pharyngitis/tonsillitis.

U.S. Pat. No. 6,268,489 claims crystalline azithromycin dihydrate. This patent discloses that the azithromycin monohydrate described in U.S. Pat. No. 4,474,768 is extremely hygroscopic and it is difficult to prepare and maintain the monohydrate product in a form having a constant, reproducible water-content. It is particularly difficult to handle during formulation, since at higher relative humidity levels the monohydrate readily picks up varying amounts of water. Such problems were overcome by the stable dihydrate, which was essentially non-hygroscopic in nature. Azithromycin currently on the market is in the form of the dihydrate.

U.S. Pat. No. 5,605,889 discloses stable formulations of azithromycin dihydrate. This patent teaches azithromycin formulations suitable for administration with food to prevent the food effect, the food effect being a major factor affecting bioavailability of the azithromycin dosage form after oral administration.

U.S. Pat. No. 6,703,372 discloses a process for the preparation of stable azithromycin monohydrate which is crystalline and which maintains its crystalline structure for at least 24 hours, e.g., for several weeks, under normal conditions, e.g., normal air humidity. The crystalline structure of azithromycin in the form of monohydrate may be determined by its known X-ray powder diffraction pattern.

WO 02/10181 discloses azithromycin monohydrate of apparently lower hygroscopicity and greater density and hardness. Such a form can be used for the preparation of stable azithromycin formulations.

There are few prior art references showing attempts to prepare stable formulations containing azithromycin monohydrate. WO04/00865 discloses pharmaceutical compositions for oral administration comprising azithromycin in the form of a monohydrate as a pharmaceutically active ingredient, a sweetener, a flavourant, a buffer, optionally a filler, and optionally a thickener. WO04/035063 also discloses orally administrable compositions comprising azithromycin that is stabilized in the form of a monohydrate.

U.S. Patent Application Nos. 2003228357, 2003190365 and 2003165563 teach formulations of azithromycin in the non-dihydrate form prepared by dry granulation, wet granulation and direct compression methods, respectively.

U.S. Pat. No. 5,633,006 claims a chewable tablet or liquid suspension pharmaceutical composition having reduced bitterness. U.S. Patent Application No. 2003176369 claims a stabilized azithromycin composition comprising an intimate admixture of azithromycin and a stabilizing-effective amount of an antioxidant. However, these attempts to stabilize azithromycin monohydrate formulations are not particularly satisfactory particularly in preventing the conversion of monohydrate into dihydrate and masking the bitter taste of the formulations.

It was observed that azithromycin monohydrate compositions prepared by wet granulation methods do not effectively prevent the conversion of monohydrate into other hydrates. The direct compression method may not be an effective method of making formulations of azithromycin monohydrate based on the compressibility of the active ingredient. We have surprisingly found that in order to prepare the stable oral azithromycin monohydrate compositions, in a sense that there is absence of other hydrates, particularly azithromycin dihydrate, it is advantageous to prepare an azithromycin “premix” which is further processed to obtained final dosage form.

### SUMMARY OF THE INVENTION

In one general aspect there is provided a stable oral composition of azithromycin that includes an azithromycin premix that includes azithromycin monohydrate and at least one additive; at least one pharmaceutically accepted excipient; and optionally, at least one taste masking agent.

Embodiments of the composition may include one or more of the following features. For example, the additive may be one or more of at least one binder, at least one disintegrant, at least one hydrophobic material, at least one surfactant, at least one lubricant, at least one diluent, and at least one taste masking agent.

The binder may be one or more of acacia, methylcellulose, carboxymethylcellulose, hydroxypropyl methylcellulose, hydroxypropylcellulose, polyvinylpyrrolidone, pregelatinized starch, gum tragacanth and sodium alginate. The disintegrant may be one or more of pregelatinized starch, sodium starch glycolate, sodium carboxymethylcellulose, crosslinked sodium carboxymethylcellulose, microcrystalline cellulose, low substituted hydroxypropyl cellulose and cross-linked polyvinylpyrrolidone. The hydrophobic material may be corn oil. The surfactant may be one or more of polysorbates, castor oil and derivatives, and sodium lauryl sulphate. The lubricant may be one or more of magnesium stearate, stearic acid, glyceryl behenate, polyethylene glycol, ethylene oxide polymers, sodium lauryl sulfate, magnesium lauryl sulfate, sodium oleate, sodium stearyl fumarate, talc, and colloidal silicon dioxide. The diluent may be one or more of lactose, sucrose, dextrose, mannitol, sorbitol, starch, microcrystalline cellulose, and dibasic calcium phosphate.

The taste masking agent may be one or more of magnesium hydroxide, magnesium carbonate, sodium carbonate, sodium phosphate, sodium citrate, calcium gluconate, meglumine, sodium chloride, sodium phosphate dibasic heptahydrate, sodium phosphate dibasic dihydrate, and anhydrous dibasic calcium phosphate.

The pharmaceutically accepted excipient may be one or more of at least one binder, at least one viscosity increasing agent, at least one disintegrant, at least one surfactant, at least one diluent, at least one lubricant, at least one dispersing agent, at least one flavoring agent, and at least one sweetening agent. The viscosity-increasing agent may be one or more of xanthan gum, guar gum, locust bean gum, gum tragacanth, alginates, sodium carboxymethylcellulose, polyvinylpyrrolidone, hydroxypropylcellulose, and hydroxypropyl methylcellulose. The flavoring agent may be one or more of menthol, flavour peppermint, flavour cherry, flavour banana, and flavour fruit gum. The sweetening agent may be one or more of aspartame, saccharin sodium, sucralose, and acesulfam K. The dispersing agent may be one or more of colloidal silicon dioxide and talc.

The composition may be prepared by a dry granulation method. The composition may be one or more of a tablet, a capsule, a powder for oral suspension, and a unit dose packet. The composition may show an absence of azithromycin dihydrate after storage at room temperature and humidity conditions for a period of at least two months, as determined by using X ray diffraction. The composition may have at least 90% dissolution of azithromycin within 30 minutes when an amount of the composition equivalent to 200 mg of azithromycin is tested according to USP-2 dissolution apparatus using 900 ml sodium phosphate buffer pH 6.0, 37° C., and paddle speed of 100 rpm.

In another general aspect there is provided a process for making a stable oral composition of azithromycin. The process includes combining azithromycin monohydrate with at least one additive to form an azithromycin premix; combining at least one pharmaceutically accepted excipient with the azithromycin premix; and optionally, adding at least one taste masking agent.

Embodiments of the process may include one or more of the following features or those features described above. For example, the additive may be one or more of at least one binder, at least one disintegrant, at least one hydrophobic material, at least one surfactant, at least one lubricant, at least one diluent, and at least one taste masking agent.

The binder may be one or more of acacia, methylcellulose, carboxymethylcellulose, hydroxypropyl methylcellulose, hydroxypropylcellulose, polyvinylpyrrolidone, pregelatinized starch, gum tragacanth and sodium alginate. The disintegrant may be one or more of pregelatinized starch, sodium starch glycolate, sodium carboxymethylcellulose, crosslinked sodium carboxymethylcellulose, microcrystalline cellulose, low substituted hydroxypropyl cellulose and cross-linked polyvinylpyrrolidone. The hydrophobic material may be corn oil. The surfactant may be one or more of polysorbates, castor oil and derivatives, and sodium lauryl sulphate. The lubricant may be one or more of magnesium stearate, stearic acid, glyceryl behenate, polyethylene glycol, ethylene oxide polymers, sodium lauryl sulfate, magnesium lauryl sulfate, sodium oleate, sodium stearyl fumarate, talc, and colloidal silicon dioxide. The diluent may be one or more of lactose, sucrose, dextrose, mannitol, sorbitol, starch, microcrystalline cellulose, and dibasic calcium phosphate. The taste masking agent may be one or more of magnesium hydroxide, magnesium carbonate, sodium carbonate, sodium phosphate, sodium citrate, calcium gluconate, meglumine, sodium chloride, sodium phosphate dibasic heptahydrate, sodium phosphate dibasic dihydrate, and anhydrous dibasic calcium phosphate.

Forming the azithromycin premix may include mixing the azithromycin monohydrate and additive. Forming the azithromycin premix may further include compacting. Forming the azithromycin premix may further include granulating.

The composition may have at least 90% dissolution of azithromycin within 30 minutes when an amount of the composition equivalent to 200 mg of azithromycin is tested according to USP-2 dissolution apparatus using 900 ml sodium phosphate buffer pH 6.0, 37° C., and paddle speed of 100 rpm. The composition may show an absence of azithromycin dihydrate after storage at room temperature and humidity conditions for a period of at least two months, as determined by using X ray diffraction.

In another general aspect there is provided a method for treating a microbial infection in a human. The method includes administering to the human a stable oral composition of azithromycin that includes an azithromycin premix that includes azithromycin monohydrate and at least one additive; at least one pharmaceutically accepted excipient; and optionally, at least one taste masking agent.

Embodiments of the method may include any one or more of the features described above.

The details of one or more embodiments of the inventions are set forth in the description below. Other features, objects and advantages of the inventions will be apparent from the description and claims.

### DETAILED DESCRIPTION OF THE INVENTION

The term “azithromycin monohydrate” as used herein refers to stable azithromycin monohydrate prepared according to U.S. Pat. No. 6,703,372 herein incorporated by reference. However any other suitable method can be used to prepared azithromycin monohydrate used in the present invention. The quantity of azithromycin monohydrate to be used in the formulation depends on the assay on anhydrous basis and water content of azithromycin monohydrate. Unless otherwise stated, the term azithromycin as used herein refers to azithromycin monohydrate.

The term “stable oral composition” as used herein and in the appended claims refers to the oral compositions of azithromycin monohydrate which are substantially free from other hydrated forms such as dihydrate. Suitable methods of determining the conversion of azithromycin monohydrate to other hydrates includes any method with substantial precision, including X-ray diffraction, IR, differential calorimetry analysis (DSC) or thermo gravimetric analysis (TGA). The water content of azithromycin monohydrate can be determined according to method of Karl Fischer. U.S. Pat. No. 6,703,372, as herein incorporated by reference, teaches a method of preparation and characterization of azithromycin monohydrate. The stable azithromycin monohydrate usefuil herein has a water content in the range of about 3% to about 8%, preferably about 4% to about 6.5% w/w.

The term “azithromycin premix” as used herein and the appended claims refer to a mixture of azithromycin monohydrate with at least one additive, preferably without additional water, in order to prevent the conversion of azithromycin monohydrate into other hydrates, particularly azithromycin dihydrate. The azithromycin premix may be obtained in the form of a powder blend, particles, granules, coated granules, compacts (e.g., slugs) or recompacts, coated compacts or coated recompacts or agglomerates which is further processed using at least one excipient to obtain azithromycin monohydrate composition in suitable dosage form.

The term “additive” refers to excipients selected from binder, diluent/filler, lubricant/glidant, disintegrant, surfactant or wetting agents, taste masking agents, hydrophobic materials, for example, corn oil, or a viscosity increasing agent, depending on the final dosage form that is being prepared.

The azithromycin premix can be further processed to obtain a final dosage form. For example, the azithromycin premix can be granulated or compacted. The granules or compacts thus obtained can be mixed with pharmaceutical accepted excipients and fuirther processed to obtain final dosage forms. The azithromycin premix can be obtained in the form of powder. The powder can be mixed with granules or compacts that are prepared using pharmaceutical accepted excipients, and further processed to obtain a final dosage form. The azithromycin premix can be further processed using wet granulation methods to obtain a final dosage form. The various methods of preparing stable oral azithromycin monohydrate composition in the form of final dosage form, using the azithromycin premix, are exemplified in the specification.

The term “composition” refers to any oral dosage form such as tablet, capsule, suspension, powder for oral suspension, unit dose packet or sachet that includes azithromycin monohydrate premix with at least one pharmaceutically accepted excipient. The pharmaceutically accepted excipient may be selected from disintegrant, binder, filler/diluent, flavoring agent, coloring agent, lubricant/glidant, sweetening agent, surfactant, dispersing agent or taste masking agent.

The “disintegrants” as used herein and in the appended claims refer to an excipient capable of swelling when in contact with water. Suitable disintegrants include starch, pregelatinized starch, sodium starch glycolate, sodium carboxymethylcellulose, crosslinked sodium carboxymethylcellulose (sodium croscarmellose; crosslinked starch available as Ac-Di-Sol® from FMC Corp., Philadelphia, Pa.), clays (e.g., magnesium aluminum silicate), microcrystalline cellulose, e.g., Avicel PH200, low substituted hydroxypropyl cellulose, alginates, effervescent mixtures, hydrous aluminum silicate, cross-linked polyvinylpyrrolidone (available commercially as PVP-XL® from International Specialty Products, Inc.), and others as known in the art. Preferred disintegrants are sodium croscarmellose (Ac-Di-Sol®), low substituted hydroxypropyl cellulose, pregelatinised starch and microcrystalline cellulose (Avicel).

Examples of binders include acacia, cellulose derivatives (such as methylcellulose and carboxymethylcellulose, hydroxypropylmethylcellulose, hydroxypropylcellulose, hydroxyethylcellulose), gelatin, glucose, dextrose, xylitol, polymethacrylates, polyvinylpyrrolidone, starch paste, sucrose, sorbitol, pregelatinized starch, gum tragacanth, alginic acids and salts thereof (such as sodium alginate), magnesium aluminum silicate, polyethylene glycol, guar gum, bentonites, and the like. For dosage forms such as powders for oral suspension and unit dose packet, the binder may also act as a viscosity-increasing agent.

A variety of materials may be used as fillers or diluents. Examples include sugars, for example, spray-dried or anhydrous lactose, sucrose, dextrose; sugar alcohol, for example, mannitol, sorbitol, xylitol, lactitol; starch, for example, starch 1500, corn starch; cellulose, for example, microcrystalline cellulose; dihydrated or anhydrous dibasic calcium phosphate (available commercially as Emcompress® from Mendell or A-Tab and Di-Tab from Rhone-Poulenc, Inc., Monmouth Junction, N.J.); calcium carbonate; calcium sulfate; corn oil and the like.

The surfactants or wetting agents may be, for example, sodium lauryl sulphate, dioctyl sodium sulfosuccinate, polyoxyethylene sorbitan fatty acid esters, castor oil derivatives, polyethylene glycol or the like.

The dispersing agent is may be, for example, colloidal silicon dioxide or talc.

The lubricant may be, for example, magnesium stearate, stearic acid, glyceryl behenate, polyethylene glycol, ethylene oxide polymers (for example, available as Carbowax® from Union Carbide, Inc., Danbury, Conn.), sodium lauryl sulfate, magnesium lauryl sulfate, sodium oleate, sodium stearyl fumarate, talc, colloidal silicon dioxide, and others as known in the art. A particularly good lubricant is magnesium stearate.

Flavoring agents incorporated in the composition may be, for example, synthetic flavor oils and flavoring aromatics and/or natural oils, extracts from plants leaves, flowers, fruits, and the like, and combinations thereof. These may include menthol, peppermint flavour, flavour cherry, flavour banana, flavour fruit gum and the like.

Coloring agents may include titanium dioxide and/or dyes suitable for food such as those known as F.D.&C, dyes and natural coloring agents such as grape skin extract, beet red powder, beta carotene, annato, carmine, turmeric, paprika, and the like. The sweetening agent may be aspartame, saccharin sodium, sucralose or acesulfam K.

In a particular embodiment, tablets of this invention are film-coated to mask the bitter taste of azithromycin and to provide an elegant appearance. Many polymeric film-coating materials are known in the art. A particularly good film-coating material is hydroxypropyl methylcellulose (HPMC). HPMC may be obtained commercially, for example, from Colorcon Corp., in coating formulations containing excipients which serve as coating aids, as Opadry®. Opadry® formulations may contain lactose, polydextrose, triacetin, polyethyleneglycol, polysorbate 80, titanium dioxide, and one or more dyes or lakes. Other suitable film-forming polymers also may be used herein, including, hydroxypropylcellulose (HPC), and acrylate-methacrylate copolymers.

Suitable viscosity increasing agents may also function as suspending agents and include, for example, hydrocolloid gums useful for such purposes, examples of which include xanthan gum, guar gum, locust bean gum, gum tragacanth, alginates and the like. Alternatively, synthetic suspending agents may be used such as sodium carboxymethylcellulose, polyvinylpyrrolidone, hydroxypropylcellulose and the like. For dosage forms such as powders for oral suspension or unit dose packet, the viscosity-increasing agent may also act as a binder.

The taste-masking agent as used herein and in the appended claims may be selected from magnesium hydroxide, sodium hydroxide, magnesium carbonate, sodium carbonate, sodium phosphate, sodium citrate, calcium gluconate, meglumine; salts such as sodium chloride; gums; anhydrous or hydrous buffering agents, for example, sodium phosphate dibasic heptahydrate/dihydrate, anhydrous dibasic calcium phosphate or the like.

The compositions of the present invention show an absence of azithromycin dihydrate after storage at room temperature and humidity conditions for the period of at least two months, as determined by X ray diffraction method. The following examples describe various embodiments of the specification and are not intended to be limiting.

### EXAMPLE 1

Azithromycin Monohydrate Tablets

| S.N. | Ingredients | 600 mg | 500 mg | 250 mg |
|---|---|---|---|---|
| | Core tablet: Stage I | | | |
| 1. | Azithromycin monohydrate* | 650.83 | 542.36 | 271.18 |
| 2. | Hydroxypropyl cellulose | 50 | 41.67 | 20.83 |
| 3. | Croscarmellose sodium | 60 | 50 | 25 |
| 4. | Sodium lauryl sulphate | 1.8 | 1.5 | 0.75 |
| | Stage II | | | |
| 5. | Pregelatinised starch | 64.8 | 54 | 27 |
| 6. | Dibasic calcium phosphate | 160 | 133.33 | 66.67 |
| 7. | Microcrystalline cellulose | 53.77 | 44.82 | 22.39 |
| 8. | Polyvinyl pyrrolidone | 20 | 16.67 | 8.33 |
| | Stage III | | | |
| 9. | Magnesium stearate | 13 | 10.83 | 5.42 |
| 10. | Croscarmellose sodium | 55 | 45.83 | 22.92 |
| 11. | Sodium lauryl sulphate | 1.8 | 1.5 | 0.75 |
| 12. | Colloidal silicon dioxide | 13 | 10.83 | 5.42 |
| 13. | Talc | 13 | 10.83 | 5.42 |
| 14. | Low substituted hydroxypropyl | 15 | 12.5 | 6.25 |
| | cellulose | | | |
| 15. | Microcrystalline cellulose | 100 | 83.33 | 41.67 |
| | Total | 1272 | 1060 | 530 |
| | Coating | | | |
| 16 | Opadry ® | 32 | 26.67 | 13.33 |
| 17 | Isopropyl alcohol and | q.s. | q.s. | q.s. |
| | Dichloromethane# | | | |

*Quantity is based on 98.6% assay on anhydrous basis and 6.5% water content.
#Does not remain in the final product, lost during drying

Procedure:
Core Tablets:

Stage I

1. Azithromycin monohydrate, hydroxypropyl cellulose, croscarmellose sodium and sodium lauryl sulphate were sifted through 30# and mixed in a blender to obtain an azithromycin premix.

2. The premix of step 1 was compacted using roll compactor.

3. The compacted material of step 2 was passed through 22#.

Stage II

4. Dibasic calcium phosphate, pregelatinised starch, polyvinylpyrrolidone and microcrystalline cellulose were sifted through 30# and mixed in a blender to obtain a powder mix.

5. The powder mix of step 4 was compacted using roll compactor.

6. The compacted material of step 5 was passed through 22#.

Staze III

7. Talc, colloidal silicon dioxide, croscarmellose sodium, sodium lauryl sulphate, hydroxypropyl cellulose and microcrystalline cellulose was sifted through 30# and mixed with material of step 3 and step 6 to obtain a blend.

8. Magnesium stearate was sifted through 44# and mixed with the blend of step 7 to obtain final blend.

9. The final blend of step 8 was compressed into tablets using suitable tooling.

Coating:

10. Opadry® is dispersed in isopropyl alcohol and dichloromethane to obtain a coating dispersion.

11. The core tablets of step 9 are coated using the coating dispersion of step 10.

### EXAMPLE 2

Azithromycin Monohydrate Tablets

| S.N. | Ingredients | 600 mg | 500 mg | 250 mg |
|---|---|---|---|---|
| | Core tablet: Stage I | | | |
| 1. | Azithromycin monohydrate | 650.83 | 542.36 | 271.18 |
| 2. | Hydroxypropyl cellulose | 50 | 41.67 | 20.83 |
| 3. | Croscarmellose sodium | 60 | 50 | 25 |
| 4. | Sodium lauryl sulphate | 1.8 | 1.5 | 0.75 |
| | Stage II | | | |
| 5. | Pregelatinised starch | 64.8 | 54 | 27 |
| 6. | Dibasic calcium phosphate | 135 | 112.5 | 56.25 |
| 7. | Microcrystalline cellulose | 59.77 | 49.81 | 24.91 |
| 8. | Magnesium hydroxide | 19.0 | 15.83 | 7.92 |
| 9. | Polyvinyl pyrrolidone | 20 | 16.67 | 8.33 |
| | Stage III | | | |
| 10. | Magnesium stearate | 13 | 10.83 | 5.42 |
| 11. | Croscarmellose sodium | 55 | 45.83 | 22.92 |
| 12. | Sodium lauryl sulphate | 1.8 | 1.5 | 0.75 |
| 13. | Colloidal silicon dioxide | 13 | 10.83 | 5.42 |
| 14. | Talc | 13 | 10.83 | 5.42 |
| 15. | Low substituted hydroxypropyl | 15 | 12.5 | 6.25 |
| | cellulose | | | |
| 16. | Microcrystalline cellulose | 100 | 83.33 | 41.67 |
| | Total | 1272 | 1060 | 530 |
| | Coating | | | |
| 17 | Opadry ® | 32 | 26.67 | 13.33 |
| 18 | Isopropyl alcohol and | q.s. | q.s. | q.s. |
| | Dichloromethane# | | | |

#Does not remain in the final product, lost during drying

Procedure:
Core Tablets:

Stage I

1. Azithromycin monohydrate, hydroxypropyl cellulose, croscarmellose sodium and sodium lauryl sulphate were sifted through 30# and mixed in a blender to obtain an azithromycin premix.

2. The premix of step 1 was compacted using roll compactor.

3. The compacted material of step 2 was passed through 22#.

Stage II

4. Dibasic calcium phosphate, magnesium hydroxide, pregelatinised starch, polyvinylpyrrolidone and microcrystalline cellulose were sifted through 30# and mixed in a blender to obtain a powder mix.

5. The powder mix of step 4 was compacted using roll compactor.

6. The compacted material of step 5 was passed through 22#.

Stage III

7. Talc, colloidal silicon dioxide, croscarmellose sodium, sodium lauryl sulphate, hydroxypropyl cellulose and microcrystalline cellulose was sifted through 30# and mixed with material of step 3 and step 6 to obtain a blend.

8. Magnesium stearate was sifted through 44# and mixed with the blend of step 7 to obtain final blend.

9. The final blend of step 8 was compressed into tablets using suitable tooling.

Coating:

10. Opadry® is dispersed in isopropyl alcohol and dichloromethane to obtain a coating dispersion.

11. The core tablets of step 9 are coated using the coating dispersion of step 10.

### EXAMPLE 3

Azitbromycin Monohydrate Tablets

| S.N. | Ingredients | 600 mg | 500 mg | 250 mg |
|---|---|---|---|---|
| | Core tablet: Stage I | | | |
| 1. | Azithromycin monohydrate | 650.83 | 542.36 | 271.18 |
| 2. | Hydroxypropyl cellulose | 50 | 41.67 | 20.83 |
| 3. | Croscarmellose sodium | 60 | 50 | 25 |
| 4. | Sodium lauryl sulphate | 1.8 | 1.5 | 0.75 |
| | Stage II | | | |
| 5. | Pregelatinised starch | 64.8 | 54 | 27 |
| 6. | Dibasic calcium phosphate | 135 | 112.5 | 56.25 |
| 7. | Microcrystalline cellulose | 59.77 | 49.81 | 24.91 |
| 8. | Calcium gluconate | 19.0 | 15.83 | 7.92 |
| 9. | Polyvinyl pyrrolidone | 20 | 16.67 | 8.33 |
| | Stage III | | | |
| 10. | Magnesium stearate | 13 | 10.83 | 5.42 |
| 11. | Croscarmellose sodium | 55 | 45.83 | 22.92 |
| 12. | Sodium lauryl sulphate | 1.8 | 1.5 | 0.75 |
| 13. | Colloidal silicon dioxide | 13 | 10.83 | 5.42 |
| 14. | Talc | 13 | 10.83 | 5.42 |
| 15. | Low substituted hydroxypropyl | 15 | 12.5 | 6.25 |
| | cellulose | | | |
| 16. | Microcrystalline cellulose | 100 | 83.33 | 41.67 |
| | Total | 1272 | 1060 | 530 |
| | Coating | | | |
| 17 | Opadry ® | 32 | 26.67 | 13.33 |
| 18 | Isopropyl alcohol and | q.s. | q.s. | q.s. |
| | Dichloromethane# | | | |

#Does not remain in the final product, lost during drying

Procedure:
Core Tablets:

Stage I

1. Azithromycin monohydrate, hydroxypropyl cellulose, croscarmellose sodium and sodium lauryl sulphate were sifted through 30# and mixed in a blender to obtain an azithromycin premix.

2. The premix of step 1 was compacted using roll compactor.

3. The compacted material of step 2 was passed through 22#.

Stage II

4. Dibasic calcium phosphate, calcium gluconate, pregelatinised starch, polyvinylpyrrolidone and microcrystalline cellulose were sifted through 30# and mixed in a blender to obtain a powder mix.

5. The powder mix of step 4 was compacted using roll compactor.

6. The compacted material of step 5 was passed through 22#.

Stage III

7. Talc, colloidal silicon dioxide, croscarmellose sodium, sodium lauryl sulphate, hydroxypropyl cellulose and microcrystalline cellulose was sifted through 30# and mixed with material of step 3 and step 6 to obtain a blend.

8. Magnesium stearate was sifted through 44# and mixed with the blend of step 7 to obtain final blend.

9. The final blend of step 8 was compressed into tablets using suitable tooling.

Coating:

10. Opadry® is dispersed in isopropyl alcohol and dichloromethane to obtain a coating dispersion.

11. The core tablets of step 9 are coated using the coating dispersion of step 10.

### EXAMPLE 4

Azithromycin Monohydrate Tablets

| S.N. | Ingredients | 600 mg | 500 mg | 250 mg |
|---|---|---|---|---|
| | Core tablet | | | |
| 1. | Azithromycin monohydrate | 650.83 | 542.36 | 271.18 |
| 2. | Pregelatinised starch | 64.8 | 54.0 | 27.0 |
| 3. | Dibasic calcium phosphate | 226.77 | 188.975 | 94.488 |
| 4. | Croscarmellose sodium | 101.6 | 84.66 | 42.33 |
| 5. | Magnesium stearate | 10.8 | 9.0 | 4.5 |
| 6. | Sodium lauryl sulphate | 3.6 | 3.0 | 1.5 |
| 7. | Colloidal silicon dioxide | 10.8 | 9.0 | 4.5 |
| 8. | Talc | 10.8 | 9.0 | 4.5 |
| | Total | 1080 | 900 | 450 |
| | Coating | | | |
| 9. | Hydroxypropyl methylcellulose | 20 | 16.66 | 8.33 |
| 10. | Triacetin | 2 | 1.66 | 0.83 |
| 11. | Talc | 2.5 | 2.09 | 1.045 |
| 12. | Titanium dioxide | 2.5 | 2.09 | 1.045 |
| 13. | Dichloromethane and isopropyl | q.s. | q.s. | q.s. |
| | alcohol# | | | |

#Does not remain in the final product, lost during drying

Procedure:
Core Tablets:

1. Azithromycin monohydrate, dibasic calcium phosphate, pregelatinised starch, part quantities of croscarmellose sodium, sodium lauryl sulphate and magnesium stearate were sifted through 30# and mixed in a blender to obtain an azithromycin premix.

2. The premix of step 1 was compacted using roll compactor.

3. The compacted material of step 2 was passed through 18#.

4. Talc, colloidal silicon dioxide, remaining quantities of croscarmellose sodium, sodium lauryl sulphate and magnesium stearate were sifted through 30# and mixed with material of step 3 to obtain a final blend.

5. The final blend of step 4 was compressed into tablets using suitable tooling.

Coating:

6. Hydroxypropyl methylcellulose, triacetin, talc and titanium dioxide were dispersed in a mixture of dichloromethane and isopropyl alcohol to obtain a coating dispersion.

7. The core tablets of step 5 were coated using the coating dispersion of step 6.

### EXAMPLE 5

Azithromycin Monohydrate Tablets 600 mg

| S.N. | Ingredients | 600 mg |
|---|---|---|
| | Core tablet | |
| 1. | Azithromycin monohydrate | 650.83 |
| 2. | Pregelatinised starch | 64.80 |
| 3. | Povidone K-30 | 20 |
| 4. | Dibasic calcium phosphate | 134.56 |
| 5. | Microcrystalline cellulose | 125.44 |
| 6. | Croscarmellose sodium | 60 |
| 7. | Hydroxypropyl cellulose-L | 50 |
| 8. | Sodium lauryl sulphate | 1.80 |
| | Extragranular | |
| 9. | Colloidal silicon dioxide | 13 |
| 10. | Microcrystalline cellulose | 66.77 |
| 11. | Low substituted hydroxypropylcellulose | 15 |
| 12. | Croscarmellose sodium | 53 |
| 13. | Sodium lauryl sulphate | 1.8 |
| 14. | Magnesium stearate | 13 |
| | Total | 1272 |
| | Coating | |
| 15. | Hydroxypropyl methylcellulose | 21.08 |
| 16. | Triacetin | 2.108 |
| 17. | Talc | 2.46 |
| 18. | Titanium dioxide | 6.15 |
| 19. | Dichloromethane and isopropyl alcohol# | q.s. |

#Does not remain in the final product, lost during drying

Procedure:
Core Tablets:

1. Azithromycin monohydrate, dibasic calcium phosphate, pregelatinised starch, croscarmellose sodium, sodium lauryl sulphate, hydroxypropylcellulose, povidone K-30 and microcrystalline cellulose were sifted through 30# and mixed in a blender to obtain a powder mix.

2. The powder mix of step 1 was compacted using roll compactor to obtain an azithromycin premix.

3. The premix of step 2 was passed through 18#.

4. Microcrystalline cellulose, low substituted hydroxypropylcellulose, croscarmellose sodium, colloidal silicon dioxide, sodium lauryl sulphate and magnesium stearate were sifted through 30# and mixed with material of step 3 to obtain a final blend.

5. The final blend of step 4 was compressed into tablets using suitable tooling.

Coating:

6. Hydroxypropyl methylcellulose, triacetin, talc and titanium dioxide were dispersed in a mixture of dichloromethane and isopropyl alcohol to obtain a coating dispersion.

7. The core tablets of step 5 were coated using the coating dispersion of step 6.

### EXAMPLE 6

Azithromycin Monohydrate Tablets 600 mg

| S.N. | Ingredients | 600 mg |
|---|---|---|
| | Core tablet | |
| 1. | Azithromycin monohydrate | 639.89 |
| 2. | Hydroxypropyl cellulose-L | 50 |
| 3. | Dibasic calcium phosphate | 239.89 |
| 4. | Pregelatinised starch | 64.8 |
| 5. | Povidone K-30 | 20 |
| 6. | Microcrystalline cellulose | 134.56 |
| | Extragranular | |
| 7. | Microcrystalline cellulose | 66.77 |
| 8. | Croscarmellose sodium | 15 |
| 9. | Hydroxypropylcellulose (LH21) | 15 |
| 10. | Colloidal silicon dioxide | 13 |
| 11. | Magnesium stearate | 13 |
| | Total | 1272 |
| | Coating | |
| 12. | Hydroxypropyl methylcellulose | 21.08 |
| 13. | Triacetin | 2.108 |
| 14. | Talc | 2.46 |
| 15. | Titanium dioxide | 6.15 |
| 16. | Dichloromethane and isopropyl alcohol# | q.s. |

#Does not remain in the final product, lost during drying

Procedure:
Core Tablets:

1. Azithromycin monohydrate, dibasic calcium phosphate, pregelatinised starch,hydroxypropylcellulose, povidone K-30 and microcrystalline cellulose were sifted through 30# and mixed in a blender to obtain an azithromycin premix.

2. The premix of step 1 was compacted using roll compactor.

3. The compacted material of step 2 was passed through 18#.

4. Microcrystalline cellulose, hydroxypropylcellulose (LH21), croscarmellose sodium, colloidal silicon dioxide and magnesium stearate were sifted through 30# and mixed with material of step 3 to obtain a final blend.

5. The final blend of step 4 was compressed into tablets using suitable tooling.

Coating:

6. Hydroxypropyl methylcellulose, triacetin, talc and titanium dioxide were dispersed in a mixture of dichloromethane and isopropyl alcohol to obtain a coating dispersion.

7. The core tablets of step 5 were coated using the coating dispersion of step 6.

### EXAMPLE 7

Azithromycin Monohydrate Tablets 600 mg

| S.N. | Ingredients | 600 mg |
|---|---|---|
| | Core tablet | |
| 1. | Azithromycin monohydrate | 639.89 |
| 2. | Hydroxypropyl cellulose-L | 100 |
| 3. | Dibasic calcium phosphate | 239.89 |
| 4. | Pregelatinised starch | 64.8 |
| 5. | Povidone K-30 | 20 |
| 6. | Microcrystalline cellulose | 84.56 |
| | Extragranular | |
| 7. | Microcrystalline cellulose | 66.77 |
| 8. | Croscarmellose sodium | 15 |
| 9. | Hydroxypropylcellulose (LH21) | 15 |
| 10. | Colloidal silicon dioxide | 13 |
| 11. | Magnesium stearate | 13 |
| | Total | 1272 |
| | Coating | |
| 12. | Hydroxypropyl methylcellulose | 21.08 |
| 13. | Triacetin | 2.108 |
| 14. | Talc | 2.46 |
| 15. | Titanium dioxide | 6.15 |
| 16. | Dichloromethane and isopropyl alcohol# | q.s. |

#Does not remain in the final product, lost during drying

Procedure:
Core Tablets:

1. Azithromycin monohydrate, dibasic calcium phosphate, pregelatinised starch,hydroxypropylcellulose, povidone K-30 and microcrystalline cellulose were sifted through 30# and mixed in a blender to obtain a powder mix.

2. The powder mix of step 1 was compacted using roll compactor to obtain an azithromycin premix.

3. The premix of step 2 was passed through 18#.

4. Microcrystalline cellulose, hydroxypropylcellulose (LH21), croscarmellose sodium, colloidal silicon dioxide and magnesium stearate were sifted through 30# and mixed with material of step 3 to obtain a final blend.

5. The final blend of step 4 was compressed into tablets using suitable tooling.

Coating:

6. Hydroxypropyl methylcellulose, triacetin, talc and titanium dioxide were dispersed in a mixture of dichloromethane and isopropyl alcohol to obtain a coating dispersion.

7. The core tablets of step 5 were coated using the coating dispersion of step 6.

### EXAMPLE 8

Azithromycin Monohydrate Powder for Oral Suspension

| S.N. | Ingredients | 200 mg/5 ml | 100 mg/5 ml |
|---|---|---|---|
| | Intragranular | | |
| 1. | Azithromycin monohydrate | 216.94 | 108.47 |
| 2. | Hydroxypropyl cellulose-L | 50 | 50 |
| 3. | Sucrose milled | 653.06 | 761.53 |
| 4. | Magnesium hydroxide | 80 | 80 |
| | Total | 1000 | 1000 |
| | Extragranular | | |
| 5. | Sucrose for granulation with sodium | 937.5 | 937.5 |
| | hydroxide | | |
| 6. | Sodium hydroxide | 15 | 15 |
| 7. | Sodium phosphate dibasic | 50 | 50 |
| | heptahydrate | | |
| 8. | Aspartame | 40 | 40 |
| 9. | Sodium chloride | 9 | 9 |
| 10. | Xanthan gum | 6 | 6 |
| 11. | Flavour Durarome cherry | 7.5 | 7.5 |
| 12. | Flavour Durarome banana | 10 | 10 |
| 13. | Flavour Peppermint | 5 | 5 |
| 14. | Menthol | 0.75 | 0.75 |
| 15. | Colour FD&C Red #40 | 1.2 | 1.2 |
| 16. | Sucrose unmilled | 1918.05 | 1918.05 |
| | Total | 4000 | 4000 |

Procedure:

1. Azithromycin monohydrate, hydroxypropyl cellulose, sucrose milled and magnesium hydroxide were sifted through 40# and mixed in a blender to obtain an azithromycin premix.

2. The premix of step 1 was compacted using roll compactor.

3. The compacted material of step 2 was passed through 40# and the fines below 60# were recompacted to obtain granules.

4. Sodium hydroxide was dissolved in purified water to obtain a solution.

5. Sucrose for granulation was granulated with the solution of step 4 above followed by drying in Fluid bed drier to obtain sucrose granules.

6. Sodium phosphate dibasic heptahydrate, sodium chloride, aspartame, xanthan gum, menthol, Durarome cherry flavour, Durarome banana flavour, peppermint flavour, Colour FD&C Red #40 and sucrose were sifted through 30# and mixed with the granules of step 3 and step 5 to obtain a final blend.

7. The final blend of step 6 was filled in a bottle.

### EXAMPLE 9

Azithromycin Monohydrate Powder for Oral Suspension

| S.N. | Ingredients | 200 mg/5 ml | 100 mg/5 ml |
|---|---|---|---|
| | Intragranular | | |
| 1. | Azithromycin monohydrate | 216.94 | 108.47 |
| 2. | Hydroxypropyl cellulose-L | 50 | 50 |
| 3. | Sucrose milled | 668.06 | 776.53 |
| 4. | Sodium hydroxide | 15 | 15 |
| 5. | Sodium phosphate dibasic dihydrate | 50 | 50 |
| | Total | 1000 | 1000 |
| | Extragranular | | |
| 6. | Aspartame | 40 | 40 |
| 7. | Sodium chloride | 9 | 9 |
| 8. | Xanthan gum | 6 | 6 |
| 9. | Flavour Durarome cherry | 10 | 10 |
| 10. | Flavour Durarome banana | 7.5 | 7.5 |
| 11. | Flavour Peppermint | 10 | 10 |
| 12. | Menthol | 0.75 | 0.75 |
| 13. | Colour FD&C Red #40 | 1.2 | 1.2 |
| 14. | Sucrose unmilled | 2915.55 | 2915.55 |
| | Total | 4000 | 4000 |

Procedure:

1. Azithromycin monohydrate, hydroxypropyl cellulose and sucrose milled were sifted through 40# and mixed in a blender to obtain an azithromycin premix.

2. Sodium hydroxide and sodium phosphate dihydrate was dissolved in purified water to obtain a solution.

3. The premix of step 1 was granulated using the solution of step 2 followed by drying in fluid bed drier to obtain granules.

4. Sodium chloride, aspartame, xanthan gum, menthol, Durarome cherry flavour, Durarome banana flavour, peppermint flavour, Colour FD&C Red #40 and sucrose were sifted through 30# and mixed with the granules of step 3 to obtain a final blend.

5. The final blend of step 4 was filled in a bottle.

### EXAMPLE 10

Azithromycin Monohydrate Powder for Oral Suspension

| S.N. | Ingredients | 200 mg/5 ml | 100 mg/5 ml |
|---|---|---|---|
| | Intragranular | | |
| 1. | Azithromycin monohydrate | 216.94 | 108.47 |
| 2. | Corn oil | 100 | 100 |
| | Extragranular | | |
| 3. | Magnesium hydroxide | 80 | 80 |
| 4. | Sucrose for granulation with sodium | 937.5 | 937.5 |
| | hydroxide | | |
| 5. | Sodium hydroxide | 15 | 15 |
| 6. | Sodium phosphate dibasic | 50 | 50 |
| | heptahydrate | | |
| 7. | Aspartame | 40 | 40 |
| 8. | Sodium chloride | 9 | 9 |
| 9. | Xanthan gum | 6 | 6 |
| 10. | Hydroxypropyl cellulose-L | 50 | 50 |
| 11. | Flavour Durarome cherry | 10 | 10 |
| 12. | Flavour Durarome banana | 7.5 | 7.5 |
| 13. | Flavour Peppermint | 10 | 10 |
| 13. | Menthol | 0.75 | 0.75 |
| 14. | Colour FD&C Red #40 | 1.2 | 1.2 |
| 15. | Sucrose unmilled | 2466.11 | 2574.58 |
| | Total | 4000 | 4000 |

Procedure:

1. Azithromycin monohydrate was sifted through 40# and mixed with corn oil to obtain an azithromycin premix.

2. Sodium hydroxide was dissolved in purified water to obtain a solution.

3. Sucrose for granulation was granulated with the solution of step 2 above followed by drying in Fluid bed drier to obtain sucrose granules.

4. Sodium phosphate heptahydrate, sodium chloride, aspartame, xanthan gum, magnesium hydroxide, hydroxypropyl cellulose, menthol, Durarome cherry flavour, Durarome banana flavour, peppermint flavour, Colour FD&C Red #40 and sucrose were sifted through 30# and mixed with the granules of step 1 & step 3 to obtain a final blend.

5. The final blend of step 4 was filled in a bottle.

### EXAMPLE 11

Azithromycin Monohydrate Powder for Oral Suspension

| S.N. | Ingredients | 200 mg/5 ml | 100 mg/5 ml |
|---|---|---|---|
| | Intragranular | | |
| 1. | Azithromycin monohydrate | 216.94 | 108.47 |
| 2. | Hydroxypropyl cellulose-L | 50 | 50 |
| 3. | Sucrose milled | 733.06 | 841.53 |
| | Total | 1000 | 1000 |
| | Extragranular | | |
| 4. | Sucrose for granulation with | 937.5 | 937.5 |
| | sodium hydroxide | | |
| 5. | Sodium hydroxide | 6 | 6 |
| 6. | Sodium alginate | 15 | 15 |
| 7. | Aspartame | 16 | 16 |
| 8. | Sodium chloride | 9 | 9 |
| 9. | Xanthan gum | 10 | 10 |
| 10. | Flavour cherry 594 SD | 7.5 | 7.5 |
| 11. | Flavour fruit gum 912 | 10 | 10 |
| 12. | Flavour Peppermint | 5 | 5 |
| 13. | Colour FD&C Red #40 | 1.2 | 1.2 |
| 14. | Titanium dioxide | 3 | 3 |
| 16. | Colloidal silicon dioxide | 8.5 | 8.5 |
| 17. | Sucrose unmilled | 1971.3 | 1971.3 |
| | Total | 4000 | 4000 |

Procedure:

1. Azithromycin monohydrate, hydroxypropyl cellulose and sucrose milled were sifted through 40# and mixed in a blender to obtain a powder mix.

2. The powder mix of step 1 was compacted using roll compactor to obtain an azithromycin premix.

3. The premix of step 2 was passed through 40# and the fines below 60# were recompacted to obtain granules.

4. Sodium hydroxide was dissolved in purified water to obtain a solution.

5. Sucrose for granulation was granulated with the solution of step 4 above followed by drying in Fluid bed drier to obtain sucrose granules.

6. Sodium chloride, aspartame, xanthan gum, sodium alginate, Flavour cherry 594 SD, flavour fruit gum 912, peppermint flavour, Colour FD&C Red #40, titanium dioxide, colloidal silicon dioxide and sucrose were sifted through 30# and mixed with the granules of step 3 and step 5 to obtain a final blend.

7. The final blend of step 6 was filled in a bottle.

### EXAMPLE 12

Azithromycin Monohydrate Powder for Oral Suspension

| S.N. | Ingredients | 200 mg/5 ml | 100 mg/5 ml |
|---|---|---|---|
| | Intragranular | | |
| 1. | Azithromycin monohydrate | 208.88 | 102.404 |
| 2. | Hydroxypropyl cellulose-L | 25 | 12.5 |
| 3. | Pregelatinised starch | 15 | 7.5 |
| | Total | 248.808 | 124.404 |
| | Extragranular | | |
| 4. | Sodium alginate | 23 | 23 |
| 5. | Xanthan gum | 4 | 4 |
| 6. | Sodium hydroxide | 6 | 6 |
| 7. | Aspartame | 16 | 16 |
| 8. | Sodium chloride | 9 | 9 |
| 9. | Flavour cherry | 7.5 | 7.5 |
| 10. | Flavour fruit gum | 10 | 10 |
| 11. | Colloidal silicon dioxide | 8.5 | 8.5 |
| 12. | Titanium dioxide | 3 | 3 |
| 13. | Colour FD&C Red #40 | 1.3 | 1.3 |
| 14. | Meglumine | 2 | 2 |
| 15. | Sucralose | 20 | 10 |
| 16. | Sucrose | 3640.892 | 3775.296 |
| | Total | 4000 | 4000 |

Procedure:

1. Azithromycin monohydrate, hydroxypropyl cellulose and pregelatinised starch were sifted through 30# and mixed in a blender to obtain an azithromycin premix.

2. The premix of step 1 was compacted using roll compactor.

3. The compacted material of step 2 was passed through 40# and the fines below 60# were recompacted to obtain granules.

4. Sodium hydroxide was dissolved in purified water to obtain a solution.

5. A part of sucrose was granulated with the solution of step 4 above followed by drying in Fluid bed drier to obtain sucrose granules.

6. Sodium alginate, sodium chloride, aspartame, xanthan gum, flavour cherry, flavour fruit gum, Colour FD&C Red #40, colloidal silicon dioxide, titanium dioxide, meglumine, sucralose and remaining quantity of sucrose were sifted through 60# and mixed with the granules of step 3 and step 5 to obtain a final blend.

7. The final blend of step 6 was filled in a bottle.

### EXAMPLE 13

Azithromycin Monohydrate Powder for Oral Suspension

| S.N. | Ingredients | 200 mg/5 ml | 100 mg/5 ml |
|---|---|---|---|
| | Intragranular | | |
| 1. | Azithromycin monohydrate | 208.88 | 102.404 |
| 2. | Hydroxypropyl cellulose-L | 25 | 12.5 |
| 3. | Pregelatinised starch | 15 | 7.5 |
| | Total | 248.808 | 124.404 |
| | Coating with ethylcellulose | | |
| 4. | Ethyl cellulose | 20 | 10 |
| 5. | Isopropyl alcohol | q.s. | q.s. |
| 6. | Methylene chloride | q.s. | q.s. |
| | Extragranular | | |
| 7. | Sodium alginate | 23 | 23 |
| 8. | Xanthan gum | 4 | 4 |
| 9. | Sodium hydroxide | 6 | 6 |
| 10. | Aspartame | 16 | 16 |
| 11. | Sodium chloride | 9 | 9 |
| 12. | Flavour cherry | 7.5 | 7.5 |
| 13. | Flavour fruit gum | 10 | 10 |
| 14. | Colloidal silicon dioxide | 8.5 | 8.5 |
| 15. | Titanium dioxide | 3 | 3 |
| 16. | Colour FD&C Red #40 | 1.3 | 1.3 |
| 17. | Meglumine | 2 | 2 |
| 18. | Sucralose | 20 | 10 |
| 19. | Sucrose | 3620.892 | 3765.296 |
| | Total | 4000 | 4000 |

Procedure:

1. Azithromycin monohydrate, hydroxypropyl cellulose and pregelatinised starch were sifted through 30# and mixed in a blender to obtain a powder mix.

2. The powder mix of step 1 was compacted using roll compactor.

3. The compacted material of step 2 was passed through 40# and the fines below 60# were recompacted to obtain granules.

4. Ethylcellulose was dissolved in isopropyl alcohol and methylene chloride to obtain a coating dispersion.

5. The granules of step 3 were coated with the coating dispersion of step 4 to obtain an azithromycin premix in the form of coated granules.

6. Sodium hydroxide was dissolved in purified water to obtain a solution.

7. A part of sucrose was granulated with the solution of step 6 above followed by drying in Fluid bed drier to obtain sucrose granules.

8. Sodium alginate, sodium chloride, aspartame, xanthan gum, flavour cherry, flavour fruit gum, Colour FD&C Red #40, colloidal silicon dioxide, titanium dioxide, meglumine, sucralose and remaining quantity of sucrose were sifted through 60# and mixed with the granules of step 5 (azithromycin premix) and step 7 to obtain a final blend.

9. The final blend of step 8 was filled in a bottle.

### EXAMPLE 14

Azithromycin Monohydrate Unit Dose Pack for Oral Suspension

| S.N. | Ingredients | 1000 mg/pack |
|---|---|---|
| | Intragranular | |
| 1. | Azithromycin monohydrate | 1084.7 |
| 2. | Hydroxypropyl cellulose-L | 250 |
| 3. | Sucrose milled | 1665.3 |
| | Total | 3000 |
| | Extragranular | |
| 4. | Sucrose for granulation with sodium hydroxide | 3750 |
| 5. | Sodium hydroxide | 60 |
| 6. | Sodium phosphate dibasic heptahydrate | 250 |
| 7. | Aspartame | 200 |
| 8. | Sodium chloride | 36 |
| 9. | Xanthan gum | 10 |
| 10. | Flavour Durarome cherry | 50 |
| 11. | Flavour Durarome banana | 100 |
| 12. | Flavour Peppermint | 50 |
| 13. | Menthol | 0.87 |
| 14. | Sucrose unmilled | 3493.13 |
| | Total | 11000 |

Procedure:

1. Azithromycin monohydrate, hydroxypropyl cellulose and sucrose milled were sifted through 40# and mixed in a blender to obtain an azithromycin premix.

2. The premix of step 1 was compacted using roll compactor.

3. The compacted material of step 2 was passed through 40# and the fines below 60# were recompacted to obtain granules.

4. Sodium hydroxide was dissolved in purified water to obtain a solution.

5. Sucrose for granulation was granulated with the solution of step 4 above followed by drying in Fluid bed drier to obtain sucrose granules.

6. Sodium phosphate dibasic heptahydrate, sodium chloride, aspartame, xanthan gum, menthol, Durarome cherry flavour, Durarome banana flavour, peppermint flavour and sucrose were sifted through 30# and mixed with the granules of step 3 and step 5 to obtain a final blend.

7. The final blend of step 6 was filled in a bottle.

### EXAMPLE 15

Azithromycin Monohydrate Unit Dose Pack for Oral Suspension

| S.N. | Ingredients | 1000 mg/pack |
|---|---|---|
| | Intragranular | |
| 1. | Azithromycin monohydrate | 1084.7 |
| 2. | Hydroxypropyl cellulose-L | 250 |
| 3. | Sucrose milled | 1355.3 |
| 4. | Sodium hydroxide | 60 |
| 5. | Sodium phosphate dibasic dihydrate | 250 |
| | Total | 3000 |
| | Extragranular | |
| 6. | Aspartame | 200 |
| 7. | Sodium chloride | 36 |
| 8. | Xanthan gum | 10 |
| 9. | Flavour Durarome cherry | 50 |
| 10. | Flavour Durarome banana | 100 |
| 11. | Flavour Peppermint | 50 |
| 12. | Menthol | 0.87 |
| 13. | Sucrose unmilled | 7553.13 |
| | Total | 11000 |

Procedure:

1. Azithromycin monohydrate, hydroxypropyl cellulose and sucrose milled were sifted through 40# and mixed in a blender to obtain an azithromycin premix.

2. Sodium hydroxide and sodium phosphate dihydrate was dissolved in purified water to obtain a solution.

3. The premix of step 1 was granulated using the solution of step 2 followed by drying in fluid bed drier to obtain granules.

4. Sodium chloride, aspartame, xanthan gum, menthol, Durarome cherry flavour, Durarome banana flavour, peppermint flavour, menthol and sucrose were sifted through 30# and mixed with the granules of step 3 to obtain a final blend.

5. The final blend of step 4 was filled in a bottle.

### EXAMPLE 16

Azithromycin Monohydrate Unit Dose Pack for Oral Suspension

| S.N. | Ingredients | 1000 mg/pack |
|---|---|---|
| | Intragranular | |
| 1. | Azithromycin monohydrate | 1084.7 |
| 2. | Corn oil | 500 |
| | Extragranular | |
| 3. | Sucrose for granulation with sodium hydroxide | 3750 |
| 4. | Sodium hydroxide | 60 |
| 5. | Sodium phosphate dibasic heptahydrate | 250 |
| 6. | Aspartame | 200 |
| 7. | Sodium chloride | 36 |
| 8. | Xanthan gum | 10 |
| 9. | Hydroxypropyl cellulose-L | 250 |
| 10. | Flavour Durarome cherry | 50 |
| 11. | Flavour Durarome banana | 100 |
| 12. | Flavour Peppermint | 50 |
| 13. | Menthol | 0.87 |
| 14. | Sucrose unmilled | 4658.43 |
| | Total | 11000 |

Procedure:

1. Azithromycin monohydrate was sifted through 40# and mixed with corn oil to obtain an azithromycin premix.

2. Sodium hydroxide was dissolved in purified water to obtain a solution.

3. Sucrose for granulation was granulated with the solution of step 2 above followed by drying in Fluid bed drier to obtain sucrose granules.

4. Sodium phosphate heptahydrate, sodium chloride, aspartame, xanthan gum, hydroxypropyl cellulose, menthol, Durarome cherry flavour, Durarome banana flavour, peppermint flavour and sucrose were sifted through 30# and mixed with the material of step 1 (azithromycin premix) and step 3 to obtain a final blend.

5. The final blend of step 4 was filled in a bottle.

### EXAMPLE 17

Azithromycin Monohydrate Unit Dose Pack for Oral Suspension

| S.N. | Ingredients | 1000 mg/pack |
|---|---|---|
| | Intragranular | |
| 1. | Azithromycin monohydrate | 1084.7 |
| 2. | Hydroxypropyl cellulose-L | 50 |
| 3. | Sucrose milled | 1865.3 |
| | Total | 3000 |
| | Extragranular | |
| 4. | Sucrose for granulation with sodium hydroxide | 1875 |
| 5. | Sodium hydroxide | 12 |
| 6. | Sodium phosphate dibasic dihydrate | 10 |
| 7. | Sodium alginate | 15 |
| 8. | Aspartame | 16 |
| 9. | Sodium chloride | 9 |
| 10. | Xanthan gum | 10 |
| 11. | Flavour cherry 594 SD | 7.5 |
| 12. | Flavour fruit gum 912 | 10 |
| 13. | Flavour Peppermint | 5 |
| 14. | Colour FD&C Red #40 | 1.2 |
| 15. | Titanium dioxide | 3 |
| 16. | Sucrose unmilled | 6026.3 |
| | Total | 11000 |

Procedure:

1. Azithromycin monohydrate, hydroxypropyl cellulose and sucrose milled were sifted through 40# and mixed in a blender to obtain a powder mix.

2. The powder mix of step 1 was compacted using roll compactor to obtain an azithromycin premix.

3. The compacted material (azithromycin premix) of step 2 was passed through 40# and the fines below 60# were recompacted to obtain granules.

4. Sodium hydroxide was dissolved in purified water to obtain a solution.

5. Sucrose for granulation was granulated with the solution of step 4 above followed by drying in Fluid bed drier to obtain sucrose granules.

6. Sodium phosphate dibasic dihydrate, sodium chloride, aspartame, xanthan gum, sodium alginate, flavour cherry 594 SD, flavour fruit gum 912, peppermint flavour, colour FD&C Red #40, titanium dioxide and sucrose were sifted through 30# and mixed with the granules of step 3 and step 5 to obtain a final blend.

7. The final blend of step 6 was filled in a bottle.

### EXAMPLE 18

Azithromycin Monohydrate Unit Dose Pack for Oral Suspension

| S.N. | Ingredients | 1000 mg/pack |
|---|---|---|
| | Intragranular | |
| 1. | Azithromycin monohydrate | 1024.03 |
| 2. | Hydroxypropyl cellulose-L | 125 |
| 3. | Pregelatinised starch | 30 |
| | Total | 1179.03 |
| | Extragranular | |
| 4. | Sodium alginate | 86 |
| 5. | Xanthan gum | 20 |
| 6. | Sodium hydroxide | 30 |
| 7. | Aspartame | 80 |
| 8. | Sodium chloride | 45 |
| 9. | Flavour cherry | 37.5 |
| 10. | Flavour fruit gum | 50 |
| 11. | Titanium dioxide | 15 |
| 12. | Colour FD&C Red #40 | 6.5 |
| 13. | Meglumine | 10 |
| 14. | Sucralose | 100 |
| 15. | Sucrose | 8340.97 |
| | Total | 10000 |

Procedure:

1. Azithromycin monohydrate, hydroxypropyl cellulose and pregelatinised starch were sifted through 30# and mixed in a blender to obtain an azithromycin premix.

2. The premix of step 1 was compacted using roll compactor.

3. The compacted material of step 2 was passed through 40# and the fines below 60# were recompacted to obtain granules.

4. Sodium hydroxide was dissolved in purified water to obtain a solution.

5. A part of sucrose was granulated with the solution of step 4 above followed by drying in Fluid bed drier to obtain sucrose granules.

6. Sodium alginate, sodium chloride, aspartame, xanthan gum, flavour cherry, flavour fruit gum, Colour FD&C Red #40, titanium dioxide, meglumine, sucrolose and remaining quantity of sucrose were sifted through 60# and mixed with the granules of step 3 and step 5 to obtain a final blend.

7. The final blend of step 6 was filled in a bottle.

### EXAMPLE 19

Azithromycin Monohydrate Unit Dose Pack for Oral Suspension

| S.N. | Ingredients | 1000 mg/pack |
|---|---|---|
| | Intragranular | |
| 1. | Azithromycin monohydrate | 1024.03 |
| 2. | Hydroxypropyl cellulose-L | 125 |
| 3. | Pregelatinised starch | 30 |
| | Total | 1179.03 |
| | Coating with ethylcellulose | |
| 4. | Ethyl cellulose | 100 |
| 5. | Isopropyl alcohol | q.s. |
| 6. | Methylene chloride | q.s. |
| | Extragranular | |
| 7. | Sodium alginate | 1279.03 |
| 8. | Xanthan gum | 20 |
| 9. | Sodium hydroxide | 30 |
| 10. | Aspartame | 80 |
| 11. | Sodium chloride | 45 |
| 12. | Flavour cherry | 37.5 |
| 13. | Flavour fruit gum | 50 |
| 14. | Titanium dioxide | 15 |
| 15. | Colour FD&C Red #40 | 6.5 |
| 16. | Meglumine | 10 |
| 17. | Sucralose | 100 |
| 18. | Sucrose | 8240.97 |
| | Total | 10000 |

Procedure:

1. Azithromycin monohydrate, hydroxypropyl cellulose and pregelatinised starch were sifted through 30# and mixed in a blender to obtain a powder mix. 2. The powder mix of step 1 was compacted using roll compactor.

3. The compacted material of step 2 was passed through 40# and the fines below 60# were recompacted to obtain an azithromycin premix in the form of granules.

4. Ethylcellulose was dissolved in isopropyl alcohol and methylene chloride to obtain a coating dispersion.

5. The granules (azithromycin premix) of step 3 were coated with the coating dispersion of step 4 to obtain coated granules.

6. Sodium hydroxide was dissolved in purified water to obtain a solution.

7. A part of sucrose was granulated with the solution of step 6 above followed by drying in Fluid bed drier to obtain sucrose granules.

8. Sodium alginate, sodium chloride, aspartame, xanthan gum, flavour cherry, flavour fruit gum, Colour FD&C Red #40, titanium dioxide, meglumine, sucralose and sucrose were sifted through 60# and mixed with the granules of step 5 and step 7 to obtain a final blend.

9. The final blend of step 8 was filled in a bottle.

While several particular forms of the inventions have been described, it will be apparent that various modifications and combinations of the inventions detailed in the text can be made without departing from the spirit and scope of the inventions. Accordingly, it is not intended that the inventions be limited, except as by the appended claims.

We claim:

1. A stable oral composition of azithromycin comprising:
an azithromycin premix comprising azithromycin monohydrate and at least one additive;
at least one pharmaceutically accepted excipient; and
optionally, at least one taste masking agent.

2. The composition of claim 1 wherein the additive comprises one or more of at least one binder, at least one disintegrant, at least one hydrophobic material, at least one surfactant, at least one lubricant, at least one diluent, and at least one taste masking agent.

3. The composition of claim 2 wherein the binder comprises one or more of acacia, methylcellulose, carboxymethylcellulose, hydroxypropyl methylcellulose, hydroxypropylcellulose, polyvinylpyrrolidone, pregelatinized starch, gum tragacanth and sodium alginate.

4. The composition of claim 2 wherein the disintegrant comprises one or more of pregelatinized starch, sodium starch glycolate, sodium carboxymethylcellulose, crosslinked sodium carboxymethylcellulose, microcrystalline cellulose, low substituted hydroxypropyl cellulose and cross-linked polyvinylpyrrolidone.

5. The composition of claim 2 wherein the hydrophobic material comprises corn oil.

6. The composition of claim 2 wherein the surfactant comprises one or more of polysorbates, castor oil and derivatives, and sodium lauryl sulphate.

7. The composition of claim 2 wherein the lubricant comprises one or more of magnesium stearate, stearic acid, glyceryl behenate, polyethylene glycol, ethylene oxide polymers, sodium lauryl sulfate, magnesium lauryl sulfate, sodium oleate, sodium stearyl fumarate, talc, and colloidal silicon dioxide.

8. The composition of claim 2 wherein the diluent comprises one or more of lactose, sucrose, dextrose, mannitol, sorbitol, starch, microcrystalline cellulose, and dibasic calcium phosphate.

9. The composition of claim 1 wherein the taste masking agent comprises one or more of magnesium hydroxide, magnesium carbonate, sodium carbonate, sodium phosphate, sodium citrate, calcium gluconate, meglumine, sodium chloride, sodium phosphate dibasic heptahydrate, sodium phosphate dibasic dihydrate, and anhydrous dibasic calcium phosphate.

10. The composition of claim 1 wherein the pharmaceutically accepted excipient comprises one or more of at least one binder, at least one viscosity increasing agent, at least one disintegrant, at least one surfactant, at least one diluent, at least one lubricant, at least one dispersing agent, at least one flavoring agent, and at least one sweetening agent.

11. The composition of claim 10 wherein the viscosity-increasing agent comprises one or more of xanthan gum, guar gum, locust bean gum, gum tragacanth, alginates, sodium carboxymethylcellulose, polyvinylpyrrolidone, hydroxypropylcellulose, and hydroxypropyl methylcellulose.

12. The composition of claim 10 wherein the flavoring agent comprises one or more of menthol, flavour peppermint, flavour cherry, flavour banana, and flavour fruit gum.

13. The composition of claim 10 wherein the sweetening agent comprises one or more of aspartame, saccharin sodium, sucralose, and acesulfam K.

14. The composition of claim 10 wherein the dispersing agent comprises one or more of colloidal silicon dioxide and talc.

15. The composition of claim 1 wherein the composition is prepared by a dry granulation method.

16. The composition of claim 1 wherein the composition comprises one or more of a tablet, a capsule, a powder for oral suspension, and a unit dose packet.

17. The composition of claim 1 wherein the composition shows an absence of azithromycin dihydrate after storage at room temperature and humidity conditions for a period of at least two months, as determined by using X ray diffraction.

18. The composition of claim 1 wherein the composition has at least 90% dissolution of azithromycin within 30 minutes when an amount of the composition equivalent to 200 mg of azithromycin is tested according to USP-2 dissolution apparatus using 900 ml sodium phosphate buffer pH 6.0, 37° C., and paddle speed of 100 rpm.

19. A process for making a stable oral composition of azithromycin, the process comprising:
combining azithromycin monohydrate with at least one additive to form an azithromycin premix;
combining at least one pharmaceutically accepted excipient with the azithromycin premix; and
optionally, adding at least one taste masking agent.

20. The process of claim 19 wherein the additive comprises one or more of at least one binder, at least one disintegrant, at least one hydrophobic material, at least one surfactant, at least one lubricant, at least one diluent, and at least one taste masking agent.

21. The process of claim 20 wherein the binder comprises one or more of acacia, methylcellulose, carboxymethylcellulose, hydroxypropyl methylcellulose, hydroxypropylcellulose, polyvinylpyrrolidone, pregelatinized starch, gum tragacanth and sodium alginate.

22. The process of claim 20 wherein the disintegrant comprises one or more of pregelatinized starch, sodium starch glycolate, sodium carboxymethylcellulose, crosslinked sodium carboxymethylcellulose, microcrystalline cellulose, low substituted hydroxypropyl cellulose and cross-linked polyvinylpyrrolidone.

23. The process of claim 20 wherein the hydrophobic material comprises corn oil.

24. The process of claim 20 wherein the surfactant comprises one or more of polysorbates, castor oil and derivatives, and sodium lauryl sulphate.

25. The process of claim 20 wherein the lubricant comprises one or more of magnesium stearate, stearic acid, glyceryl behenate, polyethylene glycol, ethylene oxide polymers, sodium lauryl sulfate, magnesium lauryl sulfate, sodium oleate, sodium stearyl fumarate, talc, and colloidal silicon dioxide.

26. The process of claim 20 wherein the diluent comprises one or more of lactose, sucrose, dextrose, mannitol, sorbitol, starch, microcrystalline cellulose, and dibasic calcium phosphate.

27. The process of claim 20 wherein the taste masking agent comprises one or more of magnesium hydroxide, magnesium carbonate, sodium carbonate, sodium phosphate, sodium citrate, calcium gluconate, meglumine, sodium chloride, sodium phosphate dibasic heptahydrate, sodium phosphate dibasic dihydrate, and anhydrous dibasic calcium phosphate.

28. The process of claim 19 wherein forming the azithromycin premix comprises mixing the azithromycin monohydrate and additive.

29. The process of claim 28 wherein forming the azithromycin premix further comprises compacting.

30. The process of claim 28 wherein forming the azithromycin premix further comprises granulating.

31. The process of claim 19 wherein the composition has at least 90% dissolution of azithromycin within 30 minutes when an amount of the composition equivalent to 200 mg of azithromycin is tested according to USP-2 dissolution apparatus using 900 ml sodium phosphate buffer pH 6.0, 37° C., and paddle speed of 100 rpm.

32. The process of claim 19 wherein the composition shows an absence of azithromycin dihydrate after storage at room temperature and humidity conditions for a period of at least two months, as determined by using X ray diffraction.

33. A method for treating a microbial infection in a human, the method comprising administering to the human a stable oral composition of azithromycin as claimed in claim 1.
